(19) United States
(12) Patent Application Publication
(10) Pub. No.: US 2010/0297234 A1
(43) Pub. Date: Nov. 25, 2010

(54) METHOD OF USING AN EXTRACELLULAR MATRIX TO ENHANCE CELL TRANSPLANT SURVIVAL AND DIFFERENTIATION

(76) Inventors: Ilene Sugino, Madison, NJ (US); Vamsi Gullapalli, Rochester, NY (US); Marco Zarbin, Chatham, NJ (US)

Correspondence Address: FOX ROTHSCHILD LLP;PRINCETON PIKE CORPORATE CENTER, 997 LENOX DRIVE, BLDG. #3, LAWRENCEVILLE, NJ, 08648, US

(21) Appl. No.: 12/738,839

(22) PCT Filed: Oct. 19, 2008
(86) PCT No.: PCT/US08/80408

§ 371 Date: Aug. 2, 2010

Related U.S. Application Data
(60) Provisional application No. 60/999,601, filed on Oct. 19, 2007.

(51) Int. Cl. A61K 9/00; C12N 5/07; A61K 35/12; C12N 5/074; C12N 5/0735; C12N 5/079; A61P 27/02

(52) U.S. Cl. 424/484; 435/377; 424/93.7

(57) ABSTRACT

Provided is a matrix for promoting survival and differentiation of cells transplanted thereon, comprising a base matrix and a cell-made matrix thereon. Methods and means for making and using same are also provided.

### RELATIONSHIP TO PRIOR APPLICATIONS

This application claims priority under 35 U.S.C. §119(e) from U.S. Provisional application No. 60/991,601 filed on Oct. 19, 2007.

### GOVERNMENTAL SUPPORT

The Research leading to the present invention was supported in part, by National Institutes of Health Grant No. NIH RO3 EY013690. Accordingly, the U.S. Government has certain rights in this invention.

### FIELD OF INVENTION

This invention relates to the production of an extracellular matrix and methods of use for clinical treatment of disease in the central nervous system.

### BACKGROUND

Disease-related changes may mask extracellular matrix ligand availability to transplanted cells, impairing post-attachment events and leading, in turn, to cell death or inability of the cells to differentiate. In addition, disease-related changes in the extracellular matrix can promote cell death, leading to the clinical situation in which cell transplantation is contemplated.

One of the conditions in which cell transplantation may be useful is age-related macular degeneration. (In addition, other conditions affecting the macula, such as retinitis pigmentosa and Stargardt disease, may benefit from cell-based therapy.) The macula lutea is an area of the retina that is about 5000 μm in diameter. The center of the macula, the fovea, contains specialized photoreceptors and provides high acuity vision necessary for reading, driving, and recognizing faces. In order for light-sensing photoreceptors to function properly, they must be in intimate contact with a cell layer called the retinal pigment epithelium (RPE). The photoreceptors and RPE exchange nutrients and other materials. The choroid is a vascular layer of the eye wall interposed between the sclera and RPE, and its capillaries, termed the choriocapillaris, provide the blood supply to the RPE and photoreceptors. The RPE is separated from the choriocapillaris by a thin layer of collagenous tissue called Bruch's membrane.

Age-related macular degeneration (AMD) is the most important cause of new cases of blindness in patients older than 55 years of age in the industrialized world. RPE cells may be one of the targets of the pathological processes that cause AMD. Approximately 10% of patients with AMD lose central vision. Among the ˜75% of AMD patients with central visual loss, abnormal blood vessels, termed choroidal new vessels (CNVs), grow from the choriocapillaris and leak fluid and blood under the RPE and macula (exudative or “wet” AMD), which causes visual loss. The stimulus for CNV growth in AMD is complex, and the biochemical pathways are now being identified. One critical element is vascular endothelial growth factor (VEGF), which is involved in CNV growth and leakage. Among ˜25% of AMD patients with severe central visual loss, the RPE and foveal photoreceptors die in the absence of CNVs (atrophic or “dry” AMD, also termed geographic atrophy (GA)). No visually beneficial treatment exits for ˜60-75% of AMD patients.

Existing therapy has significant limitations. Antioxidants, for example, do not seem to be effective in the prevention of early AMD (i.e., drusen, retinal pigmentary changes). The Age-Related Eye Disease Study (AREDS) did not show a statistically significant benefit of the AREDS vitamin and mineral formulation for either the development of new geographic atrophy or for involvement of the fovea in eyes with pre-existing geographic atrophy.

Pharmacological therapies (e.g., AVASTIN® and LUCENTIS®, both of which block the action of VEGF) that are pathway-based have provided the best treatment results for AMD patients that have ever been reported. Nonetheless, a need for improved therapy remains. Although LUCENTIS® treatment is associated with moderate visual improvement in 25-40% of patients according to the results of two randomized studies, the remaining 60-75% of patients are in urgent need of an alternative approach. Also, these medications currently are administered via repeated intravitreal injection, which entails some risk and inconvenience for the patient. Further, pharmacological therapy generally involves administration of a finite number of compounds and usually involves fluctuations in drug levels above and below the desired level.

Accordingly, novel methods and compositions are desired which would address these drawbacks of currently accepted treatment of AMD.

### SUMMARY OF INVENTION

The instant invention addresses the drawbacks of the prior art by providing, in one aspect, a modified base matrix for promoting survival and/or differentiation of target cells thereon, the modified base matrix comprising a cell-made extracellular matrix (which is a mixture of proteins and other substances) on its surface.

In different embodiments of the invention, the step of creating the cell-made extracellular matrix may be achieved by culturing, on the base matrix, the cells capable of producing such extracellular matrix, and/or by treating the base matrix with solubilized components of the extracellular matrix and/or at least an active fraction of the conditioned media from the cells capable of producing such extracellular matrix. Combination of these approaches is also contemplated.

In another aspect, the invention provides a method of increasing survival and/or differentiation of target cells on a base matrix, the method comprising: creating a cell-made extracellular matrix on the base matrix to produce a modified base matrix and administering the target cells to the modified base matrix. In different embodiments of the invention, the matrices include, without limitations, those described above.

In another aspect, the invention provides a method of increasing survival and/or differentiation of target cells on a base matrix through providing a soluble formulation of the extracellular matrix or conditioned media to the apical surface of the cells to stimulate self-assembly and deposition of extracellular matrix and/or stimulation of mechanisms for cell survival and differentiation.

In further embodiments of the invention the base matrix may be a biological matrix, such as Bruch's membrane or a synthetic polymer based matrix.

The cells capable of producing the extracellular matrix are in different embodiments selected from corneal endothelial cells, RPE cells, human embryonic stem (ES) cells and any combinations thereof. In a preferred set of embodiments, the cells are corneal endothelial cells, including, without limitations, bovine corneal endothelial cells (BCE).

In different embodiments, the target cells suitable for the methods of the instant invention are selected from RPE cells, umbilical cells, placental cells, adult stem cells, human ES cells (or other embryonic stem cells), cells derived from human ES cells (e.g. RPE derived from ES cells, retinal progenitor cells), fetal RPE cells, adult iris pigment epithelial (IPE) cells, Schwann cells, and combinations thereof. The target cells may be derived from an autologous or an allogeneic source.

In another aspect, the invention provides a conditioned media from culturing the cells capable of producing the extracellular matrix. The cells capable of forming the extracellular matrix may be the cells as described above. In a preferred embodiment, the media is collected after the cells reach confluency.

In another aspect the invention provides an active fraction of the conditioned media, as described in the previous paragraph. The active fraction is characterized by the depletion of bioactive components having molecular weight less than 20 kD, preferably less than 30 kD, more preferably, less than 50 kD, more preferably, less than 70 kD, more preferably, less than 80 kD, more preferably, less than 90 kD, and most preferably, less than 100 kD. The active fraction may also be comprised of a combination of any of the above molecular weight fractions.

In yet another aspect, the invention provides a method of treating an eye disease associated with degradation of an in situ extracellular matrix in the eye; such treatment includes creating a modified base matrix and administering the target cells to the modified base matrix.

In different embodiments of this aspect of the invention, the modified base matrix is created according to any of the embodiments of the previous aspect of the invention. Further, the target cells are chosen as described in any of the embodiments of the previous aspects of the invention.

In yet another aspect, the invention provides a kit for improving survival and differentiation of target cells on a matrix. Generally, the kit includes at least an active fraction of the conditioned media or solubilized extracellular matrix according to any embodiments described herein. The kit may also include a base matrix. In another set of embodiments, the kit comprises a modified base matrix. Further, in any embodiments of this aspect of the invention, the target cells may be provided.

In any embodiment of this aspect of the invention, suitable non-limiting examples of the base matrices, the modified base matrices, and the target cells are those described in the other aspects of the invention.

### BRIEF DESCRIPTION OF THE DRAWINGS

FIG. 1 demonstrates that long-term survival of fetal RPE on aged submacular human Bruch's membrane is impaired if the surface (basement membrane or superficial surface of the inner collagenous layer (ICL)) is not treated.

FIG. 2 demonstrates fetal RPE resurfacing on aged human submacular Bruch's membrane is improved following resurfacing with bovine corneal endothelial cell extracellular matrix (BCE-ECM).

FIG. 3 demonstrates that resurfacing aged human submacular Bruch's membrane with a biologically deposited extracellular matrix (ECM) improves long-term RPE survival compared to untreated Bruch's membrane by over 200%.

FIG. 4 illustrates RPE survival on submacular human Bruch's membrane of an AMD donor (age 79 years) cultured in serum-containing bovine corneal endothelial cell-conditioned media (BCE-CM) vs. routine RPE culture media. In the absence of Bruch's membrane treatment, these RPE cells generally show poor survival on human submacular Bruch's membrane of AMD eyes after 21 days in organ culture.

FIG. 5 illustrates improved RPE survival with 21 day exposure to serum-containing BCE-CM compared to 2 day exposure on human peripheral Bruch's membrane from a non-AMD donor (age 80 years).

FIG. 6 demonstrates that overnight treatment with serum-free BCE-conditioned media results in improvement of RPE survival and differentiation on human submacular Bruch's membrane.

FIG. 7 demonstrates that RPE derived from human embryonic stem cells successfully survive on aged human submacular Bruch's membrane treated with serum-free BCE-conditioned media (BCE-CM) (A, B), compared to untreated submacular Bruch's membrane (C, D).

FIG. 8 illustrates that soaking a polycaprolactone (PCL) scaffold in serum-free BCE-conditioned media (BCE-CM) results in improved initial RPE attachment (A) compared to no BCE-CM treatment (B).

FIG. 9 illustrates that fetal RPE attachment and survival at 5 days is improved if the RPE are initially cultured in serum-free BCE-conditioned media for 2 days.

FIG. 10 illustrates that fetal RPE cultured in RPE complete media can attach and resurface an untreated PCL scaffold, but by 7 days the cells do not exhibit density arrest.

### DETAILED DESCRIPTION

In order to alleviate the drawbacks of the prior art, cell-based therapy may offer advantages over pharmacological therapy. Cell-based therapy to replace lost or diseased RPE has the potential to preserve and restore vision in: 1) age-related macular degeneration (AMD) patients with evolving atrophy and/or choroidal neovascularization, 2) patients suffering from traumatic RPE-Bruch's membrane injury, and 3) patients with other diseases associated with RPE dysfunction (e.g., Stargardt disease and some forms of retinitis pigmentosa). In addition to replacing lost or diseased RPE with cells capable of performing RPE functions, transplanted RPE may be able to rescue nearby dying photoreceptors through their known capacity to secrete substances such as neurotrophic factors and cytokines.

As noted above, pharmacological therapy involves administration of a finite number of compounds and usually involves fluctuations in drug levels above and below the desired level. In contrast, cells placed in situ express a plethora of molecules (e.g., neurotrophic factors, cytokines) that can inhibit pathological processes and rescue neurons that are damaged by disease. Moreover, they can express these molecules in amounts, combinations, and frequencies that are tailored precisely to molecular changes that occur from moment to moment. Thus, cells have the capacity to function as “factories” that produce many more substances at appropriate doses and times than can be managed with conventional pharmacological therapy. This pharmacological salutary capacity of cell-based therapy is termed “rescue”.

Another capacity of cell-based therapy is “replacement,” which refers to the ability of transplanted cells to replace native cells that have died. In diseases such as AMD, RPE and photoreceptor cell death constitutes a component of “irreversible” visual loss in many patients. Among AMD patients with evolving atrophy, RPE transplantation could be curative.

The first efforts to develop cell-based therapy for AMD involved RPE transplantation after CNV excision. Before current pharmacological therapy was available, CNV excision was proposed as a treatment for CNVs. In most AMD patients, CNV excision is associated with iatrogenic RPE defects due to the intimate association of RPE cells and the CNV. Combined RPE transplantation and CNV excision has been attempted in AMD eyes, but it has not yet led to significant visual improvement in most patients. In contrast, RPE transplantation in animal models of retinal degeneration has been proved to rescue photoreceptors and preserve visual acuity. Although animal studies validate cell transplantation as a means of achieving photoreceptor rescue, an important distinction between humans with AMD and laboratory animals in which RPE transplantation has been successful is the age-related modification of Bruch's membrane in human eyes, which may have a significant effect on RPE graft survival.

With normal aging, human Bruch's membrane, especially in the submacular region, undergoes numerous changes (e.g., increased thickness, deposition of extracellular matrix (ECM) and lipids, cross-linking of protein, non-enzymatic formation of advanced glycation end products). These changes and additional changes due to AMD could decrease the bioavailability of ECM ligands (e.g., laminin, fibronectin, and collagen IV) and cause the poor survival of RPE cells in eyes with AMD. Thus, although human RPE cells express the integrins needed to attach to these ECM molecules, long-term transplanted RPE cell survival on aged submacular human Bruch's membrane is impaired.

Because the changes in Bruch's membrane from aging and AMD are complex and may not be fully reversible, one approach is to establish a new ECM over Bruch's membrane. Adding exogenous ECM ligands (e.g., combinations of laminin, fibronectin, vitronectin, and collagen IV) can improve RPE attachment to aged Bruch's membrane to a limited degree. (Del Piore et al., Curr Eye Res. 2002; 25:79-89). These results are consistent with the hypotheses that ECM ligand availability may decrease with Bruch's membrane aging and that it is possible to increase ligand density on this surface.

It is doubtful that attention to individual ECM ligands without attention to their 3-dimensional organization will be highly effective (as indicated by the results of previous studies). The instant disclosure demonstrates that bovine corneal endothelial cells (BCE) can attach to Bruch's membrane and, more importantly, lay down ECM. Thus, Bruch's membrane can be resurfaced with a complex ECM that is known to support excellent RPE growth and differentiation and that is well-defined biologically. (Tseng et al., J Biol. Chem. 1981; 256:3361-5; Gospodarowitz et al, J Cell Physiol. 1983; 114:191-202; Robinson et al., J Cell Physiol. 1983; 117:368-76; Nevo et al., Connect Tissue Res. 1984; 13:45-57; Sawada et al., Exp Cell Res. 1987; 171:94-109; Kay et al., Invest Opthalmol Vis Sci. 1988; 29:200-7).

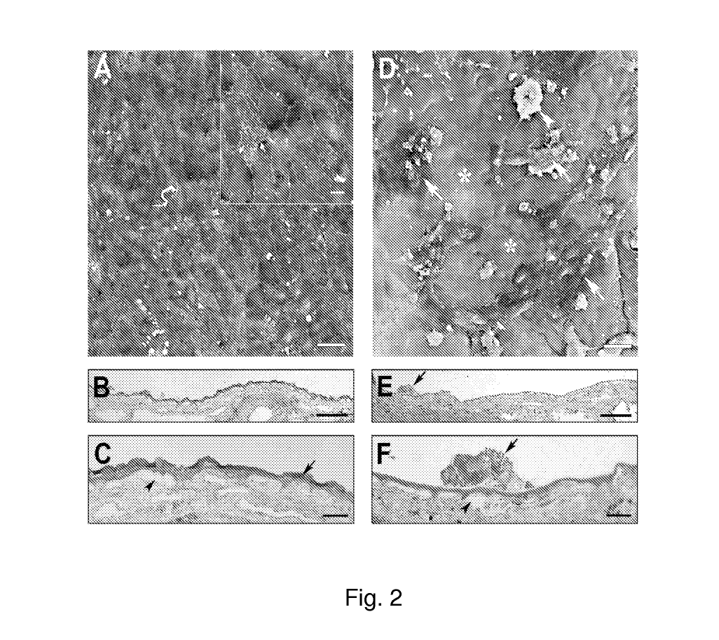

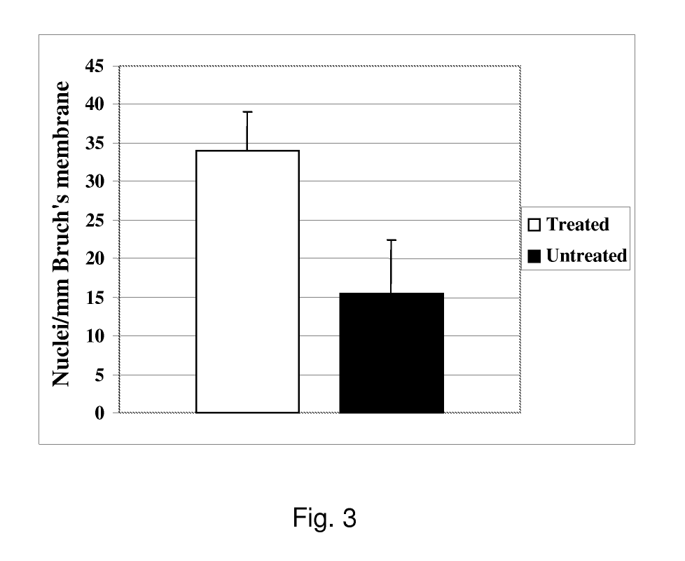

The inventors have surprisingly found that RPE focal adhesion formation on aged submacular Bruch's membrane is abnormal compared to that seen on BCE-ECM-coated culture dishes. Without wishing to be bound by any particular theory, the inventors hypothesized that this early event, probably resulting from poor ECM ligand availability, underlies later degenerative changes in RPE cells on aged Bruch's membrane after they attach. RPE focal adhesion formation is markedly improved on BCE-ECM-coated aged submacular Bruch's membrane six hours after seeding. RPE cells seeded onto the BCE-ECM-coated Bruch's membrane uniformly resurface the submacular explants with small, compact cells of variable shape. As discussed in the examples, the inventors' data demonstrate that resurfacing by BCE-ECM enhances RPE cell long-term survival on aged submacular human Bruch's membrane by ˜230% (see FIG. 2A-C and FIG. 3), which is in marked contrast to previous studies in which only modest improvement was seen following treatment with soluble ECM ligands. RPE long-term survival and differentiation are enhanced via this approach.

The research described in the instant application has demonstrated that survival of transplanted cells depends critically on the surface on which the transplanted cells grow. In two animal models, allogeneic RPE transplants can survive for at least short periods of time in the subretinal space and that freshly harvested RPE sheets or microaggregates are similarly successful. (Wang et al., Invest Opthalmol Vis Sci. 2001; 42:2990-9; Wang et al., Exp Eye Res. 2004; 78:53-65). In pigs, there is more inflammation associated with freshly harvested sheets than with cultured dispersed cell transplants, possibly due to the greater trauma associated with sheet transplantation.

AMD-related changes as well as iatrogenic changes associated with choroidal new vessel (CNV) excision create a damaged Bruch's membrane surface in eyes undergoing CNV excision. (Nasir et al., Brit. J. Opthalmol. 1997; 81:481-9; Zarbin, Arch Opthalmol. 2004; 122:598-614). In most cases, surgical damage to Bruch's membrane includes removal of the RPE basement membrane and removal of portions of the inner collagenous layer (ICL). Aged adult RPE can resurface RPE defects on aged submacular human Bruch's membrane in organ culture only to a limited extent. (Wang et al., Invest Opthalmol Vis Sci. 2003; 44:2199-2210) In addition to the surface affecting the ability of aged adult RPE to resurface RPE defects, aged RPE per se are impaired in their ability to attach and grow in culture and on Bruch's membrane. (Tsukahara et al., Exp Eye Res 2002; 74(2):255-266; Zarbin. Trans Am Opthalmol Soc 2003; 101:499-519; Wang, et al., J Rehabil Res Dev 2006; 43: 713-22; Ishida, et al., Curr Eye Res. 1998; 17: 392-402).

Resurfacing is even more limited if RPE migration/ingrowth must occur on the ICL. The in vitro wound healing data accurately predict the outcome in AMD patients following CNV removal, who show incomplete ingrowth of RPE with associated photoreceptor degeneration. (Hsu et al., Retina. 1995; 15:43-52). Freshly harvested, aged adult RPE cells, such as would be used in autologous transplants, do not survive on aged submacular Bruchs membrane. (Tsukahara et al., Exp Eye Res. 2002; 74:255-66). Culturing adult RPE cells upregulates integrins necessary for cell attachment. (Zarbin, Trans Am Opthalmol Soc. 2003; 101:499-520). However, aged adult RPE never grow as robustly on aged Bruch's membrane as young RPE. Histopathology of an AMD eye that underwent uncultured adult RPE transplantation confirms these predictions. (Del Priore et al., Am J. Opthalmol. 2001; 131:472-80).

Long-term studies of cultured fetal human RPE on aged submacular human Bruch's membrane show that many cells do not survive, and if they are present, they do not appear to be adequately differentiated. (Gullapalli et al., Exp Eye Res. 2005; 80:235-48). Of the various cell types studied to date (including adult stem cells, embryonic stem cells (differentiated into RPE-like cells), adult and fetal RPE, and adult iris pigment epithelial (IPE) cells), none appear to survive and differentiate adequately on aged submacular human Bruch's membrane. (Zarbin et al., 2003; Gullapalli et al., 2005; Gullapalli et al., Trans Am Opthalmol Soc. 2004; 102:123-37; discussion 137-8; Itaya et al., Invest Opthalmol Vis Sci. 2004; 45:4520-8).

Thus, in one aspect, the invention is drawn to a modified base matrix for survival and/or differentiation of RPE cells thereon, the modified base matrix comprising a cell-made extracellular matrix thereon.

In different embodiments, the base matrices suitable for the instant invention may be protein-based matrices, including, without limitations, collagen (including gelatin), solubilized human basement membrane, and fibrinogen-based formulations. These synthetic matrices can include mixtures optimized according to concentration of base formulations and additional cell-supporting molecules added to said formulations.

In other embodiments, the base matrices may comprise non-proteinaceous polymers, such as, for example, polycaprolactone (PCL), polylactic acid (PLA), polyglycolic acid (PGA), poly(lactide-co-glycolide) (PLGA), poly(methyl methacrylate) (PMMA), polyorthoester matrices, and any combinations thereof.

In yet another set of embodiments, the base matrices may be biological membranes, such as, for example a Bruch's membrane. In one embodiment, the Bruch's membrane used as a base matrix of the instant invention is an aged Bruch's membrane. The term “aged” essentially depends on a species source of the membrane used (e.g., assuming that the source of the membrane is human, the membrane over 40 years old, or 50 years old, or 60 years old, or 70 years old, or 80 years old, or 90 years old, or 100 years old). The species source of the Bruch's membrane include, without limitations primates, e.g., gorilla, chimpanzee, orangutan, and human. If the source of the membrane is not human, the age of the membrane should be adjusted accordingly, based on the life span of the source species.

The matrices described and/or exemplified in any of the embodiments of the invention may be located in vivo or in vitro.

The methods of production of the base templates depend on the nature of the template. For example, if the template is polymer-based (e.g., PCL based), it may be chemically synthesized. If the template is a biological membrane, as described above, it can be surgically harvested and cultured according to the methods known in the art, including, without limitations, those described in the Examples below.

Once the base matrix is chosen and obtained, it is modified with an extracellular cell-made matrix to produce a modified base matrix. The suitable cells capable of forming matrices are well known in the art and include, without limitations corneal endothelial cells (including, but not limited to, bovine cells), RPE cells (including, but not limited to, human), IPE cells (including, but not limited to, human), and stem cells (including, but not limited to, human embryonic stem cells, placental stem cells, umbilical stem cells, bone marrow-derived stem cells, neural progenitor cells).

The choice of the cells capable of forming the extracellular cell-made matrices ultimately depends on the nature of target cells which are to be grown on the modified base matrix. In a set of embodiments, wherein the target cells which are grown on the modified base matrix are RPE, corneal endothelial cells, e.g., bovine corneal endothelial cells (BCE) present a suitable option.

Another aspect of the invention is the application of conditioned media. It may be applied in one of three ways: 1) as a modification of the base matrix, 2) as a solution or in a biocompatible and degradable matrix applied to the apical surface of transplanted cells, or 3) as part of the vehicle in which the cells are transplanted.

The methods of culturing BCE cells are well known in the art although specifics of the methods may vary slightly (see, e.g., Bonanno et al., Am J Physiol Cell Physiol 277: C545-0553, 1999; Tseng et al., J. Cell Biol 1983; 97:803-809; Katz, et al, Invest Ophthal Vis Sci 1994; 35:495-502; Gospodarowicz, et al., Exp Eye Res 1977; 1:75-89; MacCallum et al., Exp Cell Res 1982; 139:1-13; Vlodaysky Curr Protocols Cell Biol 1999; 10.4.1-10.4.14. Briefly, according to the protocol published by Bonanno, the primary cultures from fresh cow eyes are established in T-25 flasks with 3 ml of DMEM, 10% bovine calf serum, and an antibiotic-antimycotic (100 U/ml penicillin, 100 μg/ml streptomycin, and 0.25 μg/ml Fungizone); gassed with 5% CO₂-95% air at 37° C., and media changed every 2-3 days. These are subcultured to three T-25 flasks and grown to confluence in 5-7 days. The resulting second-passage cultures are then subcultured onto coverslips or filters, reaching confluence within 5-7 days.

Another method of culturing BCE is to establish freshly isolated cells on tissue culture dishes (diameter 35, 60, or 100 mm) in Dulbecco's modified Eagle's medium (DMEM) supplemented with RPE complete media (DMEM with 2 mM glutamine, 15% fetal bovine serum, 2.5 μg/ml fungizone, 0.05 mg/ml gentamicin, 1 ng/ml basic fibroblast growth factor (bFGF)). Cells are grown in a humidified incubator at 10% CO₂-95% air at 37° C. until confluent with media change every 2-3 days. Upon confluency, cells are passaged at a split ratio of ˜1:3.7. First passage cells are grown in RPE complete media until confluent; second passage cells are generated by passaging first passage cells at a split ratio of ˜1:7.3.

ECM can be generated by culturing (including but not limited to) first, second, or fourth passage cells in ECM media (DMEM with 2 mM glutamine, 10% fetal bovine serum, 5% donor calf serum, 2.5 μg/ml fungizone, 0.05 mg/ml gentamicin, 1 ng/ml bFGF, 4% dextran). 1 ng bFGF is added every 2-3 days until cells are confluent. ECM can be harvested from cells at confluence or up to 3 months post-confluency. Time of ECM harvesting is specific to the cell depositing ECM. (BCE require less time to deposit ECM than RPE, including RPE derived from human ES.) Cells can be removed for ECM harvesting by exposure to 0.02M NH₄OH and/or PBS and/or detergents (e.g., 0.5% triton X-100) and/or urea (2M).

Conditioned media is generated by growing cells following passage in maintenance media (ECM media without dextran). bFGF may or may not be added every 2-3 days. 48 hours prior to collection, cells are washed a minimum of 3× in DMEM with no supplements to remove serum. Media is collected after 48 hour culturing in DMEM with no supplements.

In different embodiments of the invention, the modified base matrix may generally be created by at least three techniques: first, the matrix-forming cells are cultured on the base matrix; second, the matrix is deposited by cells onto culture dishes and harvested; and third, the matrix-forming cells are cultured separately from the base matrix, and the tissue culture media from the matrix-forming cells is collected. Harvested deposited ECM and/or media from culture may be administered to the base matrix, may be applied to the apical surface of cells, or may be used as a vehicle for cell transplantation. Apical application of the ECM and/or conditioned media can be by one of the following methods (including but not limited to): injection of the ECM and/or conditioned media solely or in a biocompatible, biodegradable matrix and/or injection following transplant cell attachment or placement onto Bruch's membrane; incorporated into the overlying material (e.g., gelatin) used for transplanting cell sheets or embedded single cells or cell aggregates. The combination of these techniques is also contemplated.

If the first or second option is employed, the matrix-formed cells may be stripped from the base matrix by chemical methods, such as, for example, NH₄OH or Urea or detergent wash or PBS soaking. Enzymatic methods (e.g., trypsin digestion) are less desirable due to possible protein damage.

If the third option is employed, it is important to keep in mind that serum, which may be present in the conditioned media, usually contains ligands of the cell-made (extracellular) matrix in the media. Accordingly, the suitable media should preferably be serum free, or at the very least, serum depleted to reduce the likelihood of inducing an inflammatory/immune response in the transplant recipient.

After sufficient time, e.g., at least 7 days or at least until cultures reach 100% confluency, or at least 1 week after confluency, or at least 2 weeks after confluency, or at least 3 months after confluency) the modified base matrix is formed to a degree sufficient to improve survival and differentiation of the cells which are to be grown on the modified base matrix (i.e., target cells). In other words, the sufficient time may be less than 3 months, or less than 2 weeks post-confluency, or less than 1 week post-confluency, or less than 7 days. As discussed above, the target cells may include, without limitations, RPE, umbilical cells, placental cells, adult stem cells, ES cells, bone marrow-derived stem cells, fetal RPEs, adult iris pigment epithelial (IPE) cells, neural progenitor cells, Schwann cells, and any combination thereof, and may be derived from an autologous or an allogeneic source.

In another aspect, the invention provides a method of increasing survival and/or differentiation of target cells on the base matrix, the method comprising: creating cell-made extracellular matrix on said base matrix to produce a modified base matrix and administering to said modified base matrix said target cells.

According to this aspect, the base matrix and the modified base matrix include, without limitations, the base matrices and the modified base matrices as described according to the previous aspect of the invention or as disclosed in the examples below.

The target cells include, without limitations, the target cells described above. In one embodiment, the cells are RPE. The RPE cells may be chosen or differentiated from multiple sources. For example, RPE may be differentiated from stem cells, such as embryonic or adult stem cells, or RPE may be fetal RPE. The methods of in vitro differentiation of RPE are known in the art.

For example, if one desires to differentiate the RPE from ES cells, US Publication 20070196919 discloses a suitable exemplary method for doing so. Briefly, the H-1 (WA-01) human embryonic stem cell line may be obtained from a commercial or a non-commercial source, such as Wicell Research Institute. The cells are cultured and passaged on a feeder layer made of irradiated mouse embryonic fibroblasts. Embryoid bodies are formed by treating undifferentiated hES colonies with 1 mg/ml of type IV collagenase (Invitrogen) and resuspending them in a 6-well ultra-low attachment plate (VWR) in the presence of media containing DMEM:F12 (Gibco), 10% knockout serum (Invitrogen), B-27 supplement (Invitrogen), 1 ng/ml mouse noggin (R&D Systems), 1 ng/ml human recombinant Dkk-1 (R&D Systems), and 5 ng/ml human recombinant insulin-like growth factor-1 (IGF-1) (R&D Systems). The cells are cultured as embryoid bodies for 3 days. On the fourth day, the embryoid bodies are plated onto poly-D-lysine-Matrigel (Collabora-tive Research, Inc)-coated plates and cultured in the presence of DMEM: F12, B-27 supplement, N-2 Supplement (Invitrogen), 10 ng/ml mouse noggin, 10 ng/ml human recombinant Dkk-1, 10 ng/ml human recombinant IGF-1, and 5 ng/ml human recombinant basic fibroblast growth factor (bFGF) (R&D Systems). The media is changed every 2-3 days.

Adult cells may also be used for creating RPE cells. For instance, retinal and corneal stem cells themselves may be utilized for cell replacement therapy in the eye. In addition, neural stem cells from the hippocampus have been reported to integrate with the host retina, adopting certain neural and glial characteristics (see review of Lund, R. L. et al., 2003, J. Leukocyte Biol. 74: 151-160). Neural stem cells prepared from fetal rat cortex were shown to differentiate along an RPE cell pathway following transplantation into the adult rat subretinal space (Enzmann, V. et al., 2003, Investig. Opthalmol. Visual Sci. 44: 5417-5422). Bone marrow stem cells have been reported to differentiate into retinal neural cells and photoreceptors following transplantation into host retinas (Tomita, M. et al., 2002, Stem Cells 20: 279-283; Kicic, A. et al., 2003, J. Neurosci. 23: 7742-7749). An ocular surface reconstruction in a rabbit model system, utilizing cultured mucosal epithelial stem cells, has also been reported.

In other embodiments, other cell types may be used for the methods of the instant invention. For example, US Publication 20050037491 (the '491 publication) reports that placental or umbilical cells injected into an eye of a dystrophic RCS rat differentiate into cells exhibiting at least some RPE characteristics, as assessed by ERG recording, rod and cone responses, a- and b-wave recording, histological examination, and Nissl staining.

In the experiments of the '491 publication, cultures of human adult umbilical and placental cells (passage-10) were expanded for 1 passage. All cells were initially seeded at 5,000 cells/cm² on gelatin-coated T75 flasks in Growth Medium. For subsequent passages, all cells were treated as follows. After trypsinization, viable cells were counted after trypan blue staining. Briefly, 50 microliters of cell suspension was combined with 50 microliters of 0.04% w/v trypan blue (Sigma, St. Louis Mo.), and the viable cell number, was estimated using a hemocytometer. Cells were trypsinized and washed three times in supplement free-DMEM:Low glucose medium (Invitrogen, Carlsbad, Calif.). Cultures of human umbilical placental and fibroblast cells at passage-11 were trypsinized and washed twice in Leibovitz's L-15 medium (Invitrogen, Carlsbad, Calif.). For the transplantation procedure, dystrophic RCS rats were anesthetized with xylazine-ketamine (1 mg/kg i.p. of the following mixture: 2.5 ml xylazine at 20 mg/ml, 5 ml ketamine at 100 mg/ml, and 0.5 ml distilled water), and their heads secured by a nose bar. Cells devoid of serum were resuspended (2×10⁵ cells per injection) in 2 microliters of Leibovitz, L-15 medium (Invitrogen, Carlsbad, Calif.) and transplanted using a fine glass pipette (internal diameter 75-150 micrometers) trans-sclerally. Cells were delivered into the dorso-temporal subretinal space of anesthetized 3 week old dystrophic-pigmented RCS rats (total N=10/cell type).

As discussed throughout this disclosure, treatment of the base matrix with a conditioned media from BCE cells is sufficient for improved survival and/or differentiation of the target cells. Accordingly, in another aspect, the invention provides a conditioned media from cultured cells capable of producing the cell-made matrix, according to any embodiment, as described above. In addition, the inventors have surprisingly discovered that experiments with media harvested from passage-2 cultures show that media harvested from cells that have been in culture for 2 weeks after reaching confluency is not as supportive as media harvested at earlier time points (50% confluent, confluent, 1 week after confluency). Thus, in a preferred embodiment, the conditioned culture medium is harvested from the cells that have not been confluent for more than 2 weeks.

The inventors have also discovered that the whole conditioned media is not necessary for the improved survival and/or differentiation of the RPE on the modified base matrix. Thus, in another aspect, the invention is drawn to the active fraction of the conditioned culture media, according to any of the embodiments described above. Specifically, the inventors have found that high molecular weight components are sufficient for the initial beneficial effect of the conditioned culture media. Specifically, such an active fraction may be characterized by having its low molecular weight components depleted. However, low molecular weight components may be important in long-term survival and differentiation.

In different embodiments, the active fraction is characterized by the depletion of bioactive components having molecular weight less than 20 kD, preferably less than 30 kD, more preferably, less than 50 kD, more preferably, less than 70 kD, more preferably, less than 80 kD, more preferably, less than 90 kD, and most preferably, less than 100 kD. The active fraction may be characterized by any combination of components separated according to size or other methods (e.g., high pressure liquid chromatography (HPLC)).

The depletion of low molecular weight or other non-essential components may be achieved by many methods, including, without limitation, filtration, size fractionation by gel filtration or gradient centrifugation, HPLC (separation according to charge, size, or hydrophobicity), immunoprecipitation, affinity column separation, and the like. However, it is important that the methods of depletion of low-molecular weight compounds should not result in protein cleavage nor should it disrupt secondary and tertiary protein structures of any needed components in the medium.

While it is possible to surgically remove CNVs, CNV excision is associated with iatrogenic RPE defects due to the intimate association of RPE cells and the CNV. (Thomas et al., Am J Opthalmol 1991; 111:1-7; Nasir et al., Br J Opthalmol 1997; 81:481-489; Castellarin et al., Retina 1998; 18:143-149; Hsu et al., Retina 1995; 15:43-52; Rosa et al., Arch Opthalmol 1996; 114:480-487). Combined RPE transplantation and CNV excision has been attempted in AMD eyes, but it has not led to significant visual improvement in most patients. (Algvere et al., Graefes Arch Clin Exp Opthalmol 1994; 232:707-716; Del Priore et al., Am J Opthalmol 2001; 131:472-480; Binder et al., Am J Opthalmol 2002; 133:215-225; Tezel et al., Am J Opthalmol 2007; 143:584-595; Joussen et al., Am J Opthalmol 2006; 142:17-30). Potential causes of RPE transplant failure in human patients include immune rejection, inability of transplanted RPE cells to survive and differentiate on aged submacular Bruch's membrane, and choriocapillaris atrophy, all causing death of the RPE graft. In contrast, RPE transplants rescue photoreceptors and preserve visual acuity in animal models of retinal degeneration. (Li et al., Exp Eye Res 1988; 47:911-917; Coffey et al., Exp Neurol 1997; 146:1-9; Lund et al., Proc Natl Acad Sci USA 2001; 98:9942-9947; Wang et al., Invest Opthalmol Vis Sci 2008; 49:416-421; Gias et al., The European journal of neuroscience 2007; 25:1940-1948).

An important distinction between humans with AMD and laboratory animals is the age-related modification of Bruch's membrane that occurs in human eyes. With normal aging, human Bruch's membrane, especially in the submacular region, undergoes numerous changes (e.g., increased thickness, deposition of extracellular matrix (ECM) and lipids, cross-linking of protein, nonenzymatic formation of advanced glycation end products). (Guymer et al., Prog Retin Eye Res 1999; Marshall et al., The Retinal Pigment Epithelium. New York: Oxford University Press; 1998:669-692; 18:59-90; Abdelsalam et al., Surv Opthalmol 1999; 44:1-29). Pauleikhoff and coworkers reported an age-related decline in the presence of laminin, fibronectin, and collagen IV in the RPE basement membrane. (Pauleikhoff et al., Opthalmologe 2000; 97:243-250). It is possible that changes in submacular Bruch's membrane permeability and choriocapillary density may contribute to age-related RPE death. However, we have found that RPE survival is also impaired on aged submacular Bruch's membrane explants in organ culture, where diffusion of nutrients is not a factor in cell survival. This finding suggests that there are additional factors within aged Bruch's membrane itself that adversely affect RPE survival and that modification of Bruch's membrane may have a significant effect on RPE graft survival in patients with AMD. (Gullapalli et al., Exp Eye Res 2002; 74:255-266).

As discussed above and shown in the examples below, in the instant invention, the use of modified base matrix according to any embodiment of the invention promotes survival and/or differentiation of cells transplanted onto this matrix. In embodiments where the base matrix is an aged Bruch's membrane or a Bruch's membrane from an eye undergoing macular degeneration, survival and/or differentiation of transplanted RPE was improved when the Bruch's membrane was modified with extracellular matrix from BCE cells. Importantly, the experiments were performed in human eyes, thus validating the methods and compounds of the instant invention for human treatment.

Accordingly, in one aspect, the methods according to the instant inventions may be performed for treatment of humans suffering from AMD (whether the wet AMD or the dry AMD). The terms “treat” or “treatment” or “treating” etc., refer to executing a protocol in an effort to alleviate signs or symptoms of a disease. Alleviation may occur either before or after appearance of these signs or symptoms. In addition, these terms do not require a complete alleviation of the signs or symptoms, do not require a cure, and include protocols resulting in only marginal effects on a patient.

Specifically, the methods of treatment comprise modifying Bruch's membrane with the cell made extracellular matrix, according to any embodiments described herein, and wherein the Bruch's membrane is located in vivo. Essentially, in different embodiments, Bruch's membrane is modified when the at least the active fraction of the conditioned media (or the whole conditioned media) of any of the embodiments described above or exemplified below can be applied basally as a substrate to coat the surface of Bruch's membrane, in a mixture with cells, or apically in a biocompatible matrix.

It is also worth noting that this invention has been shown to support adult and embryonic stem cells and retinal pigment epithelial cells (adult and fetal) on human Bruch's membrane, including Bruch's membrane from AMD eyes. Accordingly, in different embodiments, different types of cells may be applied within the methods of this aspect of the invention.

The compositions containing the extracellular matrix (e.g., at least the active fraction of the conditioned media according to any embodiment of the instant invention) can be applied to Bruch's membrane in living patients through a variety of strategies, e.g., direct application to the subretinal space.

In another embodiment, the scaffold (i.e., the base matrix), such as, for example, a polymeric scaffold such as PCL, can be delivered into the subretinal space. In different embodiments, the scaffold is modified with the extracellular matrix (resulting in the modified base matrix), as described above and exemplified below. Further, such modified base matrix may be delivered in combination with a scaffold that contains cells to be transplanted to the patient's eye. The suitable cells have been described above.

In another aspect, the invention provides a kit for treatment of AMD (both wet AMD and dry AMD). Generally, the kit would include a set of instructions and at least the active fraction of the conditioned media, as described in any of the embodiments of the instant invention, and may comprise the unfractionated conditioned media, also, according to any of the embodiments of the instant invention. Alternatively, the kit may comprise the cells capable of producing the cell-made extracellular matrix, according to any of the embodiments of the instant invention. Specifically and without limitations, the cells capable of producing the cell-made extracellular matrix include BCE cells. Alternatively, the kit may include ECM generated and harvested from cell-deposited matrices in solubilized or non-solubilized form.

Optionally, the kit may provide the base matrix, according to the embodiments described above. The base matrix may be a natural polymer (e.g., a protein-based base matrix), a synthetic polymer (e.g., PCL), a biological membrane (e.g., Bruch's membrane), or a combination thereof.

In another set of embodiments, the kit may comprise a modified base matrix, according to any of the embodiments described herein. In any of the embodiments of the kit, suitable target cells may also be provided, according to any of the embodiments described above.

The set of instructions may be provided in any media, including, without limitations, written, graphic, audio recording, video recording, and electronic media.

The invention will now be described in the following non-limiting examples.

### EXAMPLES

### Example 1

### Long-Term Survival of Fetal RPE on Aged Submacular Human Bruch's Membrane is Impaired

Fetal RPE (3164 cells/mm²) were seeded on aged human submacular Bruch's membrane debrided to expose the superficial surface of the inner collagenous layer. To create surfaces exposing the RPE basement membrane, RPE were gently wiped off the RPE/choroid/sclera explant using a wet surgical sponge. To create surfaces exposing the surface of the inner collagenous layer beneath the RPE basement membrane (i.e., superficial ICL), following RPE removal as indicated previously, a moistened surgical sponge was use to abrade the RPE basement membrane. In general, the area of RPE basement membrane debridement was created by approximately 5 wipes of the moistened sponge in each of 4 directions (rotating the explant 90 degrees after each series of 5 wipes). (V. K. Gullapalli, et al., Exp Eye Res 2005; 80(2):235-248). Cells were seeded onto the sclera/choroid explant and cultured for 21 days and evaluated for resurfacing with scanning electron microscopy (SEM) and light microscopy (LM). Nuclear density counts (mean±SD) of fetal RPE on aged submacular human Bruch's membrane at day-1 (basement membrane, N=7; superficial ICL, N=7), day-7 (basement membrane, N=6; superficial ICL N=6), day-14 (basement membrane N=7), day-21 (superficial ICL, N=6) were performed on 5 non-adjacent slides in the central 3 mm of the section (includes the submacular region of Bruch's membrane). Cells on tissue culture dishes coated with BCE-ECM (N=1) are included for comparison. Cells were seeded at a density of 3164 cells/mm² for all time points and surfaces.

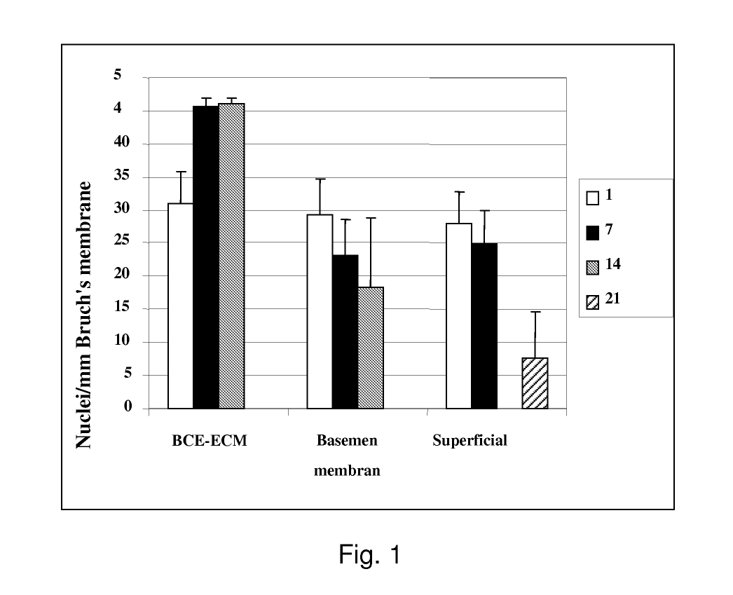

Fetal RPE survival on submacular Bruch's membrane decreased with time, regardless of the surface on which the cells are seeded (e.g., RPE basement membrane or the surface of the inner collagenous layer (superficial ICL)) (See FIG. 1, modified from V. K. Gullapalli, et al., Exp Eye Res 2005; 80(2):235-248.) (Transplanted RPE will encounter superficial ICL in situ if native RPE are removed by CNV excision.) In contrast, density increased to 45 nuclei/mm² if RPE are grown on bovine corneal endothelial cell extracellular matrix (BCE-ECM)-coated culture dishes.

### Example 2

### Fetal RPE Resurfacing on Aged Bruch's Membrane Resurfaced with Bovine Corneal Endothelial Matrix (BCE-ECM)

BCE (3164 cells/mm²) were cultured on the inner collagenous layer of aged human submacular Bruch's membrane (65 yr. old donor) for 14 days to allow ECM deposition. Cells were culture in the same way as cells cultured for ECM deposition on culture dishes (see paragraph 0056). Following BCE removal with NH₄OH to expose the newly deposited ECM and extensive washing with PBS, explants were seeded with fetal RPE (3164 cells/mm²) and cultured for 21 days. The results of these experiments are illustrated in FIG. 2.

FIG. 2A is a scanning electron micrograph (SEM), showing that fetal RPE fully resurfaced the treated explant with large, flat polymorphic cells. Cells showed varying amounts of short apical processes on their surfaces (insert). Mag. bar 50 μm; insert mag bar 10 μm.

FIGS. 2B and 2C are light micrographs (LMs). As shown in FIG. 2B, cells fully resurfaced the treated explant and are in a monolayer. Mag. bar 100 μm. FIG. 2C is a higher magnification of the explant shown in FIG. 2B, allowing one to discern the variable morphology of the cells. Cells are tightly adherent to the explant surface. Arrow in FIG. 2C points to the nucleus of a cell in the monolayer; arrowhead to a choriocapillaris vessel. Mag. bar 20 μm.

As a negative control, submacular Bruch's membrane of the fellow eye was incubated in serum-free media with no BCE for 14 days followed by exposure to NH₄OH, rinsing with PBS and fetal RPE seeding and culturing for 21 days.

FIG. 2D is a SEM of the RPE on the untreated Bruch's membrane surface. Notably, fetal RPE incompletely resurfaced the untreated inner collagenous layer. Islands of large, flattened cells are present (arrows). Dead, dying, or poorly attached cells are also present on the surface or attached to the flattened cells (arrowhead). Asterisk, exposed inner collagenous layer surface. Mag. bar 50 μm.

FIGS. 2 E and 2F show a representative view of the RPE on untreated Bruch's membrane. In this section, there is only a single clump of cells (arrow). Mag. bar 100 μm. FIG. 2F is a high magnification of the clump of cells shown in FIG. 2E. Arrow points to a cell in the clump that is not intact. Arrowhead points to a choriocapillaris vessel. Mag. bar 20 μm.

### Example 3

### Resurfacing Bruch's Membrane with a Biologically Deposited Extracellular Matrix (ECM) Improves Cell Survival

Bovine corneal endothelial cells (BCE, passage-2) were seeded onto human submacular superficial ICL of Caucasian donors over 55 years old at a density of 3164 cells/mm² and cultured for 14 days to allow ECM deposition or treated for days with serum-free media only. Following BCE removal with NH₄OH and extensive rinsing, fetal RPE (passage-2-5) were seeded at the same density onto the treated Bruch's membrane surface and cultured for 21 days. The fellow eye was treated similarly except no BCE were seeded.

RPE seeding density was 3164 cells/mm² for 21 day incubations to determine long-term survival and morphology. FIG. 3 shows the cumulative data from 9 explant pairs. Counts are mean fetal RPE nuclei/mm Bruch's membrane (±SEM).

A statistically significant 230% (p=0.006) increase in cell density is seen at day-21 on treated explants compared to explants treated with serum-free DMEM only (FIG. 3) or explants in which cells were seeded directly on Bruch's membrane with no prior treatment (FIG. 1, day-21 superficial ICL (striped bar)).

### Example 4

### Bovine Corneal Endothelial Cells (BCE) Secrete ECM Components into the Overlying Media

During ECM formation, in addition to basal secretion, BCE secrete ECM components into the media (BCE-conditioned media, BCE-CM), and the composition and relative amounts of the components vary with culture time and passage number. Secretion of ECM components into the overlying media is most abundant in early passage cells (up to passage-2) and exceeds basal ECM deposition in quantity. (Tseng et al. J Biol Chem 1981; 256:3361-3365).

Serum-free BCE-conditioned media (BCE-CM) was prepared from passage-2 cells that were cultured in serum-free Dulbecco's modified Eagle's medium (DMEM) for 48 hrs. An initial sample concentrated using a 30 kD cut-off filter identified 20 proteins by MS/MS-MALDI. The proteins in an additional sample of conditioned media, unfiltered, were subjected to 2D LC-MS/MS, and samples were analyzed with MALDI-TOF and QTOF.

These analyses identified 84 proteins (at least one peptide having C.I. values of >95%). Conditioned media from the same preparation was also analyzed by 2D gel separation, and selected spots (142) were analyzed by MALDI-TOF. This analysis identified 45 different proteins. A combined total of 109 proteins were identified using these methods (Table 1).

TABLE 1
Protein components of two different samples of bovine corneal endothelial cell
conditioned media (BCE-CM) as determined by MS/MS-MALDI, LC-MS/MS
(MALDI-TOF and Q-TOF), and MS/MS-MALDI of selected 2D gel spots.
Proteins were identified based on at least one peptide with C.I. value 95% or more.

| Name | Protein Description |
|---|---|
| IGF-1 | prepro-insulin-like growth factor I |
| IGFBP-2 | Insulin-like growth factor-binding protein-2 |
| IGFBP-4 | insulin-like growth factor-binding protein-4 |
| IGFBP-7 | PREDICTED: similar to insulin-like growth factor binding protein 7 (predicted), |
| | partial |
| FN | Fibronectin (FN) |
| hypothetical | hypothetical protein LOC504471 |
| protein | |
| H factor 1 | H factor 1 (complement) |
| C3 | Chain B, Structure Of Mammalian C3 With An Intact Thioester At 3a Resolution |
| C3 | Complement component 3 |
| C3d | Complement component C3d |
| | PREDICTED: similar to Complement C3 precursor, partial |
| | Complement C3 precursor [Contains: Complement C3 beta chain; Complement C3 |
| | alpha chain; C3a anaphyl |
| C4 | PREDICTED: similar to Complement C4 precursor |
| CCP modules | CCP modules 3-12, with parts of CCP 2 and 13 |
| collagen, type I | collagen, type I, alpha 2 |
| collagen, type III | collagen, alpha-1 (III) chain |
| collagen, type V | type V preprocollagen alpha 2 chain |
| collagenase type | collagenase type IV precursor |
| IV | |
| TIMP2 | tissue inhibitor of metalloproteinase 2 |
| MMP2 | matrix metalloproteinase 2 |
| EGF-containing | PREDICTED: similar to EGF-containing fibulin-like extracellular matrix protein 1 |
| fibulin-like | isoform a precursor |
| extracellular | |
| matrix protein 1 | |
| | PREDICTED: similar to EGF-containing fibulin-like extracellular matrix protein 1 |
| | isoform b |
| fibulin-3, FIBL-3 | EGF-containing fibulin-like extracellular matrix protein 1 precursor |
| fibulin-1 | fibulin-1 C |
| SPARC protein | SPARC protein |
| Osteonectin | secreted protein, acidic, cysteine-rich (osteonectin) |
| ESM-1 | PREDICTED: similar to Endothelial cell-specific molecule 1 precursor (ESM-1 |
| | secretory protein) |
| apolipoprotein A | apolipoprotein A-I precursor |
| apolipoprotein E | apolipoprotein E |
| | apolipoprotein E precursor |
| EC-SOD | PREDICTED: similar to Extracellular superoxide dismutase [Cu—Zn] precursor (EC- |
| | SOD) isoform 2 |
| PCSK9 | PREDICTED: similar to proprotein convertase subtilisin/kexin type 1 inhibitor |
| | precursor |
| alpha-actin | alpha-actin |
| Alpha-cardiac | PREDICTED: similar to Actin, alpha cardiac (Alpha-cardiac actin) isoform 1 |
| actin | |
| Beta-actin | Actin, cytoplasmic 1 (Beta-actin) |
| actinin | PREDICTED: similar to actinin alpha 4 isoform 3 |
| | actinin, alpha 1 |
| POTE-2 | PREDICTED: similar to Prostate, ovary, testis expressed protein on chromosome 2 |
| Dkk-3 | PREDICTED: similar to Dickkopf related protein-3 precursor (Dkk-3) (Dickkopf-3) |
| | (hDkk-3) |
| | dickkopf homolog 3 |
| cathepsin L | cathepsin L |
| Fibulin-1 | PREDICTED: similar to Fibulin-1 precursor isoform 1 |
| Thrombospondin-1 | Chain A, Crystal Structure Of The Thrombospondin-1 N-Terminal Domain |
| Vimentin | Vimentin |
| PGDS | prostaglandin D2 synthase precursor |
| ITI | PREDICTED: similar to inter-alpha trypsin inhibitor heavy chain precursor 5 isoform 1 |
| nidogen | nidogen |
| Entactin | PREDICTED: similar to Nidogen precursor (Entactin) isoform 3 |
| Osteonidogen | PREDICTED: similar to Nidogen-2 precursor (NID-2) (Osteonidogen) |
| MGP | matrix Gla protein |
| HSPG | PREDICTED: heparan sulfate proteoglycan 2 |
| | heparan sulfate proteoglycan perlecan |
| | PREDICTED: similar to Basement membrane-specific heparan sulfate |
| | proteoglycan core protein precursor |
| nephronectin | PREDICTED: similar to nephronectin isoform b |
| FSTL1 | Follistatin-related protein 1 precursor (Follistatin-like 1) (TGF-beta-inducible protein |
| | TSC-36) |
| FSTL3 | PREDICTED: similar to Follistatin-related protein 3 precursor (Follistatin-like 3) |
| | (Follistatin-rel |
| LTBP-2 | Latent-transforming growth factor beta-binding protein 2 precursor (LTBP-2) |
| albumin | albumin |
| transthyretin | transthyretin |
| NPC | Alveolar macrophage chemotactic factor (Neutrophil chemotactic protein) (NPC) |
| CTGF | connective tissue growth factor |
| Dimeric Bovine | Chain B, Dimeric Bovine Tissue-Extracted Decorin, Crystal Form 2 |
| Tissue-Extracted | |
| Decorin | |
| GOLPH2 | PREDICTED: similar to golgi phosphoprotein 2 |
| KIAA1133 | PREDICTED: similar to KIAA1133 protein |
| GST | glutathione S-transferase, GST {N-terminal} {EC 2.5.1.18} [cattle, erythrocytes, |
| | Peptide Partial, 2 |
| serine protease | serine protease |
| SMC1 | mitosis-specific chromosome segregation protein SMC1 homolog |
| superfast myosin | PREDICTED: similar to superfast myosin heavy chain |
| AEBP1 | AE binding protein 1 |
| angiomodulin | angiomodulin |
| anti-PPS | anti-pneumococcal capsular polysaccharide immunoglobulin heavy chain variable |
| | region |
| N-cadherin | Cadherin-2 precursor (Neural-cadherin) (N-cadherin) (CD325 antigen) |
| CD44 | CD44 antigen precursor (Phagocytic glycoprotein I) (PGP-1) (HUTCH-I) |
| | (Extracellular matrix receptor |
| APT | Chain A, Crystal Structure Of The First Active Autolysate Form Of The Porcine |
| | Alpha Trypsin |
| ACTH | preproadrenocorticotropic hormone (ACTH) |
| | corticotropin |
| | corticotropin-like interm lobe peptide |
| cystatin C | cystatin C (amyloid angiopathy and cerebral hemorrhage) [Bos taurus] |
| fibromodulin | fibromodulin |
| galanin | galanin |
| polypeptide | precursor polypeptide (AA −31 to 1139) |
| PTGDS | prostaglandin H2 D-isomerase |
| microglobulin | similar to beta 2-microglobulin |
| lumican | lumican |
| gelsolin | gelsolin |
| cadherin 11 | cadherin 11, type 2 preproprotein |
| Transferrin | Serotransferrin precursor (Transferrin) (Siderophilin) (Beta-1-metal-binding |
| | globulin) |
| PDI | Protein disulfide-isomerase A3 precursor (Disulfide isomerase ER-60) (ERP60) |
| Nucleobindin 1 | Nucleobindin 1 |
| Calnuc | Chain A, Nmr Solution Structure Of The Calcium-Binding Domain of Nucleobindin |
| | (Calnuc) |
| Osteonectin | Secreted protein, acidic, cysteine-rich |
| Beta-globin | Hemoglobin subunit beta (Hemoglobin beta chain) (Beta-globin) |
| Alpha enolase | Alpha enolase |
| FHOS2S splicing | FHOS2S splicing variant |
| variant [ | |
| Aldehyde | aldehyde dehydrogenase family 1, subfamily A1 |
| dehydrogenase | |
| LDHA | Lactate dehydrogenase-A |
| ATIC-ALK | Tropomyosin 4-anaplastic lymphoma kinase fusion protein |
| | Tyrosine 3-/tryptophan 5-monooxygenase activation protein, epsilon polypeptide |
| Human Annexin | Chain A, Structure Of Human Annexin A2 In The Presence Of Calcium Ions |
| A2 | |
| Peroxiredoxin 1 | Peroxiredoxin 1 |
| Peroxiredoxin 6 | Peroxiredoxin 6 |
| Anti-oxidant | Anti-oxidant protein 2 (non-selenium glutathione peroxidase, acidic calcium- |
| protein 2 | independent phospholipa) |
| NME4 | Expressed in non-metastatic cells 1 protein |
| biglycan | biglycan |
| clusterin | clusterin |
| | osteoglycin |
| | plasminogen activator inhibitor type 1, member 2 |
| | transketolase |

### Example 5

### Bovine Corneal Endothelial Cell Conditioned Media (BCE-CM) Can Improve RPE Cell Survival on Aged Human Submacular Bruch's Membrane

BCE-CM containing serum was prepared by exposing newly confluent cultures of BCE to RPE complete media (DMEM with 2 mM glutamine, 15% fetal bovine serum, 2.5 μg/ml fungizone, 0.05 mg/ml gentamicin, 1 ng/ml bFGF) for 3 days. Media was centrifuged and supernatant stored frozen. Submacular aged human Bruch's membrane explants were debrided to expose the superficial inner collagenous layer; 3164 cells/mm² were seeded on each explant and cultured for 21 days. Explants with cells were cultured in serum-containing BCE-CM or RPE complete media.

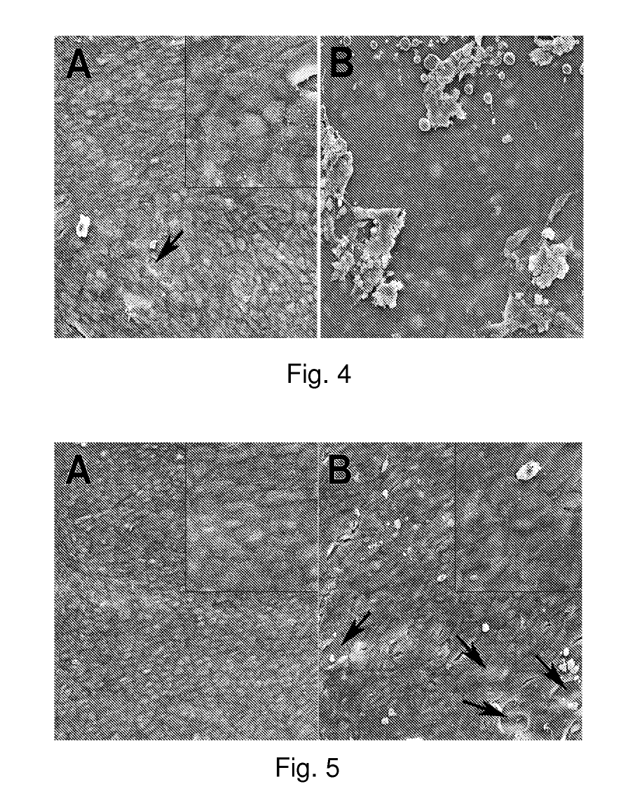

Preliminary data using serum-containing BCE-CM as media for RPE following seeding onto peripheral (N=2) and submacular Bruch's membrane (N=1, with submacular drusen) shows BCE-CM used as media supports better RPE attachment and long-term survival than RPE complete media (FIG. 4). In the experiments illustrated in FIG. 4, submacular human Bruch's membrane of an AMD donor (age 79 years) was treated with serum-containing bovine corneal endothelial cell-conditioned media (BCE-CM) vs. routine RPE culture media; BCE-CM was prepared by exposing passage-2 BCE for 3 days in RPE complete media containing serum. The explant to be treated had a greater number of large submacular drusen than the control explant, which means it was the more severely diseased of the two eyes. A. RPE cultured in BCE-CM, day 21. Cells fully resurface the treated explant with a few small defects (arrow). High magnification insert shows short apical processes on the surface of some cells and along cell borders. B. RPE cultured in RPE complete media, day-21. The explant is sparsely resurfaced with patches or clumps of RPE. Original magnifications 200×; insert 1000×.

The inventors have also shown serum-containing BCE-CM used as media during the duration of the incubation (21 days) showed better cell morphology and resurfacing on peripheral inner collagenous layer of Bruch's membrane than explants where BCE-CM was changed to standard RPE media (which also contains serum) after 2 days (FIG. 5). In these experiments, RPE survival on peripheral Bruch's membrane from a non-AMD donor (age 80 years) was investigated. A. Aged human peripheral Bruch's membrane explant cultured for 21 days in BCE-CM. Explant is fully resurfaced with a fairly uniform monolayer of cells. High magnification insert shows short apical processes covering the surface of the cells. B. Aged human peripheral Bruch's membrane (isolated from fellow eye of that shown in A) explant cultured for 2 days in BCE-CM then placed in RPE complete media for 19 days. Explant is incompletely resurfaced (arrows point to defects) with cells of varying size and morphology. High magnification insert shows the cells have a few very short apical processes, and the cells are fairly large and smooth. Original magnifications: 200×; inserts: 1000×.

### Example 6

### Bovine Corneal Endothelial Cell Conditioned Media (BCE-CM) Supports Rapid RPE Attachment and Spreading on Non-Tissue Culture Treated Plastic to a Similar Degree as Cells on BCE-ECM-Coated Tissue Culture Plastic

Soluble ECM can affect cell shape and metabolism in addition to stimulating production of ECM molecules. The inventors performed studies to determine: 1) whether soluble components in BCE-CM can be used instead of BCE-ECM to coat culture dishes and support fetal RPE growth and differentiation; and 2) whether BCE-CM used as media for cell suspension and seeding can support cells on non-tissue culture treated dishes (NTC). Since serum contains ECM ligands (e.g., vitronectin and fibronectin), these studies were performed in serum-free media as the most stringent test of cell support. Because of RPE dependence on serum in the media for long-term survival, experiments were performed for 3 days only.

Serum-free conditioned media (sfBCE-CM) was prepared from passage-2 cultures as described above. sfBCE-CM was applied in media or by coating non-tissue culture treated dishes (NTC) unconcentrated or in concentrated form (8-fold, using a 30 kD cut-off filter). Negative control was cells seeded and cultured in DMEM only. Fetal RPE (passage-3) were seeded at a density of 526 cells/mm² for all attachment studies. To determine whether non-protein components of BCE-CM contribute to early attachment and spreading of fetal RPE, sfBCE-CM was heated to 80° for 15 minutes, centrifuged, and the supernatant was used as media for attachment and seeding of fetal RPE. The importance of intact protein components in BCE-CM evidenced by cell behavior in heat-treated sfBCE-CM was confirmed by treatment with proteinase K agarose beads (removed prior to cell suspension and seeding) before and after heat treatment.

sfBCE-CM used either as media (Table 2, A) or as a substrate to coat tissue culture dishes (Table 2, B) supported rapid RPE adhesion and cell division in serum-free conditions. sfBCE-CM-treated dishes supported rapid attachment and spreading by 1 hour (Table 2, B), similar to BCE-ECM-treated dishes (Table 2, D). Fetal RPE seeded in heat inactivated and/or proteinase K-treated BCE-CM behaved similar to those on NTC (Table 2A, C). Cells seeded onto 8× sfBCE-CM did attach and spread but to a slightly lesser degree than on unconcentrated CM. The best morphology (uniform spreading, less filopodia formation) was observed in cells on BCE-ECM and in sfBCE-CM used as media or as a coating substrate. Experiments are in progress to determine whether differences in cell behavior are observed in media harvested from BCE of different passages and times in culture.

Preliminary experiments with media harvested from passage-2 cultures show that media harvested from cells that have been in culture for 2 weeks after reaching confluency is not as supportive as media harvested at earlier time points (50% confluent, confluent, 1 week after confluency) (data not shown). Protein composition analysis is currently underway to determine changes in the media harvested at these different time points to determine what proteins may account for the decreased cell support.

TABLE 2
Fetal RPE behavior in serum-free BCE conditioned media (sfBCE-CM) under
different culture conditions. Cells were seeded at the same density for all experiments. A.
Effect of sfBCE-CM as media for attachment and growth. sfBCE-CM, heat inactivated sfBCE-
CM, and sfBCE-CM treated with proteinase K are compared. B. Effect of sfBCE-CM as a
surface treatment for attachment and growth on non-tissue culture treated (NTC) dishes, either
unconcentrated or concentrated 8X using a 30 kD cut-off filter. Cells were suspended and
cultured in either DMEM or sfBCE-CM. C. Control (cells on untreated NTC dishes with
DMEM as media). D. Control (cells on BCE-ECM with DMEM as media).

| | | | |
|---|---|---|---|
| Experimental | | | |
| Condition | 1 Hour | Day-1 | Day-3 |

A. RPE CELL ATTACHMENT AND GROWTH IN sfBCE-CM

| | | | |
|---|---|---|---|
| sfBCE-CM | ~40% | ~70-80% spread, almost | Confluent, uniform cell |
| | spread | confluent | size, few vacuoles |
| sfBCE-CM, heat | Rounded | Few cells, filopodia | More cells than at day-1 but |
| inactivated | | | very few, poor morphology. |
| sfBCE-CM, proteinase | Rounded | Rounded | Rounded |
| K treatment with and | | | |
| without prior heat | | | |
| inactivation | | | |

B. sfBCE-CM SURFACE COATING OF NTC DISHES FOLLOWED BY RPE SEEDING
WITH DMEM OR sfBCE-CM MEDIA

| | | | |
|---|---|---|---|
| sfBCE-CM, DMEM | 40-50% | High density, confluent in | Confluent, small cells, |
| | spread | center, some multi- | good, morphology, few |
| | | nucleate cells | vacuoles |
| sfBCE-CM, sfBCE- | ~70% | Moderate density, | Elongate cells with |
| CM | spread | filopodia and lamellipodia | filopodia and lamellipodia, |
| | | | subconfluent |
| 8X sfBCE-CM, | ~60% | High density of cells, | Almost confluent, more |
| MW > 30K, DMEM | spread | confluent in center, | variable morphology than |
| | | similar to 1X BCE-CM, | 1X BCE-CM, DMEM |
| | | DMEM | |
| 8X sfBCE-CM, | 50-60% | Moderate density of cells, | Elongate cells with |
| MW > 30K, sfBCE-CM | spread | filopodia and lamellipodia | filopodia and lamellipodia, |
| | | | subconfluent |

C. NEGATIVE CONTROL

| | | | |
|---|---|---|---|
| NTC plastic, DMEM | Rounded | Rounded | Few elongated cells |

D. POSITIVE CONTROL

| | | | |
|---|---|---|---|
| BCE-ECM surface | ~50-90% | Majority cells are spread | Cells spread and |
| | spread | but not confluent, some | proliferating, almost |
| | | filopodia | confluent |

### Example 7

### Soaking in Serum-Free BCE-CM can Improve Cell Survival on Aged AMD Bruch's Membrane

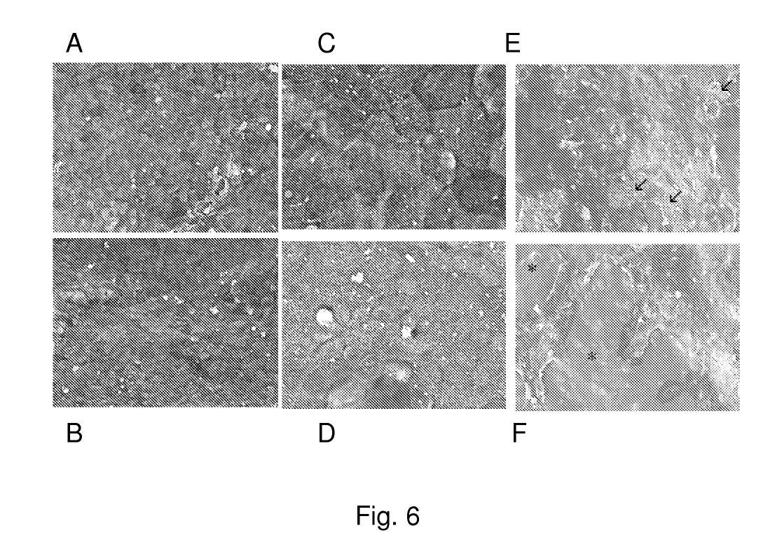

FIG. 6 demonstrates that even a relatively short treatment (i.e., overnight) leads to improvement in RPE survival on and resurfacing of Bruch's membrane. In this experiment, submacular Bruch's membrane from an 80 year-old Caucasian male donor was debrided to expose the superficial inner collagenous layer. Large submacular drusen were present on Bruch's membrane of both eyes, and the eye treated with BCE-CM showed more deposits (i.e., was the more severely diseased of the two eyes). Bruch's membrane was treated by overnight soaking of the explant in serum-free BCE-CM; the fellow eye explant was soaked for the same period of time in regular serum-free media (DMEM). Fetal RPE were seeded onto both Bruch's membrane explants at a seeding density of 3164 cells/mm². Both explants were cultured in RPE complete media for 21 days.

The explant treated with the conditioned media as described in the previous paragraph shows almost 100% resurfacing with a few small defects (FIGS. 6A, B, original magnification 200×). The high magnification images (1000×) show small cells with varying amounts of apical processes (a differentiation feature) (FIGS. 6C, D). In contrast, the untreated explant shows incomplete resurfacing by very large flat smooth cells (FIG. 6 E, F, original magnification 200×). Areas of cellular debris are evident where the cells have died (arrows). Asterisks indicate areas not resurfaced.

### Example 8

### Treatment of Aged Bruch's Membranes with BCE Conditioned Media Improves Survival of RPE Derived from Human ES Cells

In order to investigate whether treatment of surfaces with BCE-conditioned media improves survival and/or differentiation of RPE other than fetal RPE, the following experiments were performed. Fresh (not frozen) RPE derived from human ES cells (hES-RPE obtained from Advanced Cell Technology, Inc.) of intermediate pigmentation were seeded onto the inner collagenous layer of submacular Bruch's membrane from a 63 year-old Caucasian female at a seeding density of 3164 cells/mm². There was no evident pathology in the macula of either eye. The treated explant was cultured in serum-containing BCE-CM while the untreated explant was cultured in RPE complete media. Explants were harvested after 21 days in culture.

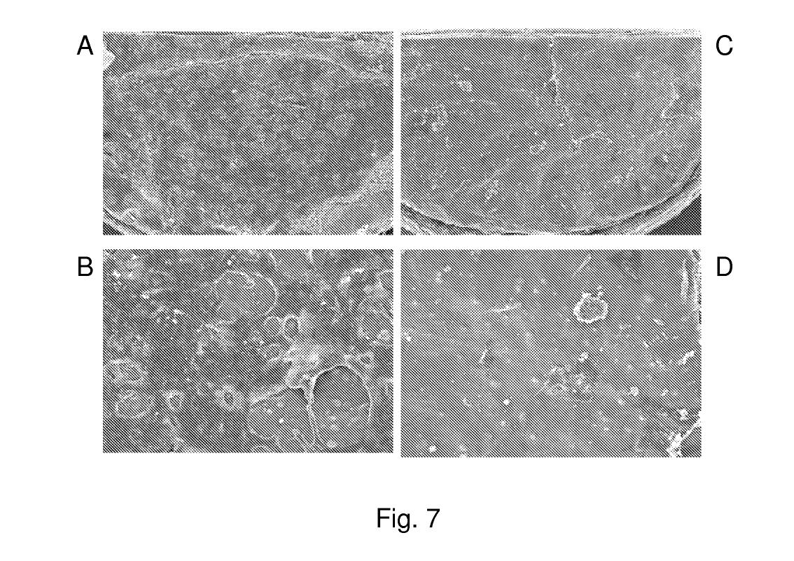

The results of these experiments are illustrated in FIG. 7. The treated explant (FIG. 7 A, B) demonstrated some degree of resurfacing by the hES-RPE with defects in coverage. The cells were very flat and did not show prominent differentiated features. The untreated explant (FIGS. 7C, D) showed only sparse coverage, with only a few cells in the submacular region of the explant. Thus, BCE-CM treatment improved hES-RPE survival on aged human Bruch's membrane.

### Example 9

### The Active Components in BCE-CM Appear to be of Molecular Weight (MW) 30 kD and Higher

Different preparations of sfBCE-CM of different molecular weight cut-off were prepared to determine the MW fraction of active components in sfBCE-CM. Media were reconstituted to 1× following filtration. Retentate solutions of MW>3 kD, >10 kD, >30 kD, and >50 kD and filtrate solutions of MW<3 kD, <10 kD, <30 kD, and <50 kD were prepared. RPE (passage-4) were suspended in each solution and seeded onto non-tissue cultured treated plastic (NTC) as detailed above. In a separate study, sfBCE-CM was filtered using a 100 kD molecular cut off filter, yielding filtrates of <100 kD and retentates of >100 kD. Fetal RPE (passage-3) behavior was observed in the 2 solutions up to day-2.

The active cell-supporting components in BCE-CM appear to be of molecular weight (MW) 30 kD and higher. Based on day-3 observations of vacuole formation (early apoptotic changes) in RPE cultured in retentate fractions containing proteins of molecular weight less than 30 kD, it appears that proteins present in the low molecular weight fractions may have a negative effect on the cells. Molecular weight fractions of 100 kD and higher supported rapid initial RPE attachment in serum-free media. Molecular weight fractions below 100 kD did not support rapid attachment and spreading in serum-free media to any degree.

Thus, it appears that high molecular weight fractions (>100 kD) are important in initial RPE attachment and spreading in serum-free conditions.

TABLE 3
Fetal RPE behavior in serum-free BCE conditioned media of different molecular
weights. RPE were seeded at the same density for all experiments. The effects
of sfBCE-CM as media for attachment and growth, prepared by centrifugal
filtration of different MW cut-offs (retentates above MW 3, 10, 30, 50, 100 kD and
filtrates below MW 3, 10, 30, 50, 100 kD) are shown.

| Molecular weight | | | |
|---|---|---|---|
| fraction of sfBCE- | | | |
| CM | 1 Hour | Day-1 | Day-3 |
| Low MW (<3, 10, 30, | Rounded | Rounded | Rounded |
| or 50 kD) | | | |
| High MW (>3, 10, 30, | 30-40% | >3 kD confluent, >10, 30, | All confluent, >3 kD |
| or 50 kD) | spread | or 50 kD almost confluent | smallest cells with most |
| | | to confluent with | vacuoles; >10 kD vacuoles, |
| | | intercellular gaps | uniform cell size; >30 kD |
| | | | less vacuoles, uniform cell |
| | | | size; >50 kD mixed sizes, |
| | | | no vacuoles |
| sfBCE-CM (unfiltered | 30-40% | Confluent with | Confluent, mixed sizes, few |
| control for above | spread | intercellular gaps | vacuoles |
| studies) | | | |
| >100 kD | ~70% | ~90% spread, some | No observations |
| | attached and | filopodia (more than | |
| | spread | seen in cells on BCE- | |
| | | ECM) | |
| <100 kD | 80-90% | 20-30% spread, others | No observations |
| | attached, | are round, abundant | |
| | round | filopodia | |
| sfBCE-CM (unfiltered | ~70% | ~90% spread, some | No observations |
| control for 100 kD cut- | attached and | filopodia | |
| off studies) | spread | | |

### Example 10

### BCE Conditioned Media is Effective at Dilutions Up to 20×

Fetal RPE (passage-3, 526 cells/mm²) were seeded onto non-tissue culture treated plastic in dilutions of serum-free BCE conditioned media (sfBCE-CM, 1:1 to 1:80 dilutions) to determine the maximum effective dilution of BCE-CM for support of initial RPE attachment and spreading. Negative control was cells seeded in serum-free DMEM. Results (Table 4). Support of attachment and spreading was seen in BCE-CM diluted up to 1:10 in serum-free DMEM. Cells in 1:20 and higher dilutions show increasingly poor attachment and morphology at day-1 after seeding.

TABLE 4
Fetal RPE behavior in diluted serum-free BCE-conditioned media.
Fetal RPE were suspended in different dilutions of serum-free
BCE-CM and seeded onto non-tissue culture treated dishes.

| Dilution of | | |
|---|---|---|
| sfBCE-CM | 1 Hour | Day-1 |
| 1:1 | ~60-70% attached and | ~90-95% attached and well spread |
| | spread | |
| 1:5 | ~60-70% attached and | ~90-95% attached and well spread |
| | spread | |
| 1:10 | ~50-55% attached and | ~90% attached and well spread |
| | spread | |
| 1:20 | ~50 attached and | ~60% attached, not as well spread |
| | spread | as higher concentrations. Cells |
| | | aggregated. |
| 1:40 | ~30-40% attached and | ~30-40% attached, variably spread. |
| | spread | Cells aggregated; variable |
| | | morphology with lamellipodia, |
| | | filopodia. Some cells elongated, |
| | | some not spread. |
| 1:80 | <5% attached and | <10% attached, some elongated |
| | spread | minimal spreading. Cells |
| | | aggregated; all of poor morphology |
| | | with elongation, filopodia and |
| | | lamellipodia. |
| DMEM | Rounded, few spread | All rounded. |
| (negative | | |
| control) | | |

### Example 11

### RPE can Attach and Grow on PCL Scaffolds

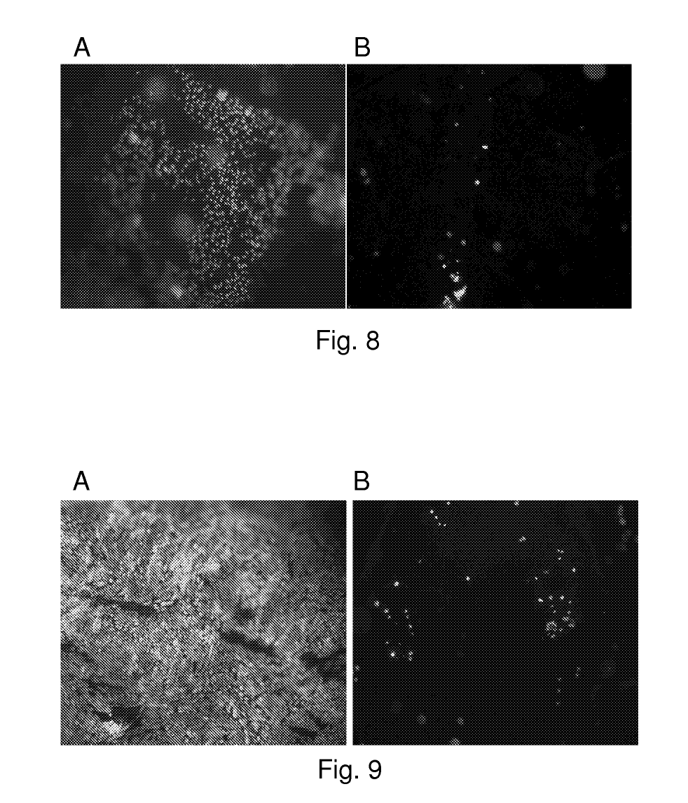

1052 fetal RPE/mm² were seeded onto 5 mm diameter PCL scaffolds and cultured for 1 day. To assess attachment onto the scaffolds, cell behavior was compared on scaffolds with no treatment (FIG. 8, B) vs. scaffold soaked in serum-free BCE conditioned media (sfBCE-CM, soaked for ˜1 hr. at 37° C.) to allow protein adsorption (FIG. 8, A). Cells were cultured in DMEM or in sfBCE-CM. RPE were visualized on the scaffolds with calcein imaging. RPE appeared to attach only to scaffolds treated with or cultured in sfBCE-CM (Table 5, 1 day and FIG. 8). Greatest attachment and spreading were observed in cells seeded onto sfBCE-CM-soaked scaffolds.

To determine whether cells could eventually adhere and spread on the scaffolds, scaffolds were exposed to sfBCE-CM by either soaking (FIG. 9, A), followed by cell seeding and culturing in DMEM for 2 days or using sfBCE-CM as media for 2 days. Controls were cells on untreated scaffolds in DMEM for 2 days (FIG. 9, B). Cultures were changed to RPE complete media (DMEM with 2 mM glutamine, 15% fetal bovine serum, 2.5 μg/ml fungizone, 0.05 mg/ml gentamicin, 1 ng/ml bFGF) after day 2 and cultured for 3 days. RPE were able to resurface the scaffolds only if the scaffold was pre-soaked in sfBCE-CM or sfBCE-CM was used as media for two days (see FIG. 9 and Table 5, 5 days).

To determine whether untreated scaffolds could support eventual resurfacing by RPE, we examined cell behavior on untreated scaffolds that were cultured in RPE complete media for 7 days. Cells were seeded at the same density we seed cells onto Bruch's membrane (3164 cells/mm²).

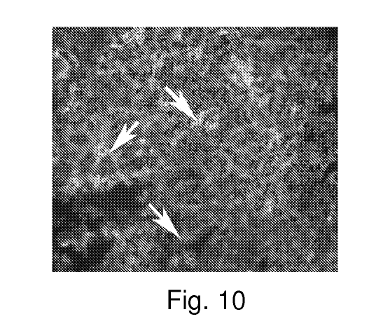

RPE fully resurfaced the untreated scaffold although the cells did not appear to density arrest by this time point (FIG. 10, arrows point to areas of multilayer formation). We observe similar multilayer formation in RPE seeded onto tissue culture plastic and onto glass coverslips.

PCL scaffolds can support initial fetal RPE attachment and resurfacing if exposed to sfBCE-CM as a substrate coating the scaffold or as media overlying seeded cells. Although untreated scaffolds may support long-term survival of RPE in serum-containing media, modification of the scaffold or addition of ECM ligands may be necessary to support differentiated cell monolayers.

TABLE 5
Fetal RPE behavior on PCL scaffolds that are either untreated or soaked in
serum-free BCE conditioned media (sfBCE-CM). For 1 day studies, cells
on untreated scaffolds were cultured in sfBCE-CM or DMEM; cells on
sfBCE-CM-soaked scaffolds were cultured in DMEM. For 5 day studies,
cells were cultured on untreated or sfBCE-CM-soaked scaffolds for 2 days
in DMEM or sfBCE-CM followed by media change to RPE complete media.
For 7 day studies, untreated scaffolds were cultured in RPE complete media.
Cell behavior on BCE-ECM-coated culture dishes (no scaffold controls) is
included for day-1 and day-7 data for comparison.

| Time in | | | |
|---|---|---|---|
| Culture | Scaffold Treatment | Media | Cell Behavior |
| 1 day | None | DMEM | Few rounded cells |
| | None | sfBCE-CM | Many cells, many are spread |
| | sfBCE-CM | DMEM | Many cells, many are spread |
| | BCE-ECM on plastic | DMEM | Many cells, majority are spread |
| 5 days | None | 2d DMEM, 3d RPE | Few rounded cells |
| | | complete media | |
| | None | 2d sfBCE-CM, 3d | Fully resurfaced |
| | | RPE complete | |
| | | media | |
| | sfBCE-CM | 2d DMEM, 3d RPE | Fully resurfaced |
| | | complete media | |
| 7 days | None | RPE complete | Fully resurfaced, some multi- |
| | | media | layering |
| | BCE-ECM on plastic | RPE complete | Fully resurfaced, monolayer |
| | | media | |

All publications cited in the specification, both patent publications and non-patent publications, are indicative of the level of skill of those skilled in the art to which this invention pertains. All these publications are herein fully incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

1. A method of increasing survival and/or differentiation of target cells on a base matrix, the method comprising:
creating a cell-made extracellular matrix on said base matrix to produce a modified base matrix and
administering said target cells to said modified base matrix.

2. The method of claim 1, wherein the step of creating the cell-made extracellular matrix on said base matrix to produce the modified base matrix is performed in vitro.

3. The method of claim 1, wherein the step of administering said target cells to said modified base matrix is performed in vivo.

4. The method of claim 1, wherein the step of creating the cell-made extracellular matrix on said base matrix to produce the modified base matrix is performed in vivo.

5. A modified base matrix for survival and/or differentiation of target cells thereon, the modified base matrix comprising a cell-made extracellular matrix thereon.

6. The modified base matrix of claim 5, wherein the modified base matrix is produced by culturing cells capable of forming said cell-made extracellular matrix on the base matrix.

7. The modified base matrix of claim 5, wherein said modified base matrix is produced by treating the base matrix with at least an active fraction of a conditioned media from culturing cells capable of forming said cell-made extracellular matrix on the base matrix.

8. The modified matrix of claim 5 wherein said base matrix is Bruch's membrane.

9. The modified base matrix of claim 8, wherein the Bruch's membrane is undergoing macular degeneration.

10. The modified matrix of claim 5, wherein said base matrix is a synthetic polymer-based matrix.

11. The modified base matrix of claim 10, wherein the synthetic polymer is selected from polylactic acid (PLA), polyglycolic acid (PGA), poly(lactide-co-glycolide) (PLGA), poly(methyl methacrylate) (PMMA), polyorthoesters, and any combinations thereof.

12. The modified base matrix of claim 11, wherein the synthetic polymer is polycaprolactone (PCL).

13. The modified base matrix of claim 5, wherein said target cells are selected from RPE, umbilical cells, placental cells, adult stem cells, embryonic stem cells, fetal RPE, adult iris pigment epithelial (IPE) cells, bone marrow-derived stem cells, Schwann cells, neural progenitor cells, and any combination thereof.

14. The modified base matrix of claim 5, wherein said target cells are autologous.

15. The modified base matrix of claim 5, wherein said target cells are fetal RPEs.

16. The modified base matrix of claim 5, wherein the target cells are adult or embryonic stem cells or are differentiated from adult or embryonic stem cells.

17. A conditioned media from culturing cells capable of forming a cell-made extracellular matrix on a base matrix on the modified base matrix of claim 5.

18. The conditioned media of claim 17, which is serum-free.

19. The conditioned media of claim 17, wherein said cells capable of forming said cell-made extracellular matrix on the base matrix are selected from corneal endothelial cells, RPE cells, IPE cells, embryonic stem cells, bone marrow-derived stem cells, placental cells, and/or umbilical cells.

20. The conditioned media of claim 19, wherein said cells are corneal endothelial cells.

21. The conditioned media of claim 19, wherein the conditioned media or at least a fraction thereof is produced by culturing cells capable of forming said cell-made extracellular matrix.

22. An active fraction of a conditioned media of claim 17, characterized by a depletion of biologically active components having MW of less than about 100 kD.

23. The conditioned media of claim 17, wherein the active fraction of the conditioned media from culturing cells capable of forming said cell-made extracellular matrix on the base matrix is formed by a depletion of biologically active components having MW of less than about 100 kD.

24. A kit for promoting the survival and/or differentiation of target cells on a base matrix, comprising:
a) a set of instructions and at least one of:
b) an efficient amount of cells capable of forming a cell-made extracellular matrix; and
c) at least an active fraction of a conditioned media or an extracellular matrix from the cells capable of forming a cell-made extracellular matrix.

25. (canceled)

26. A kit for promoting survival and differentiation of target cells comprising a set of instructions and a modified base matrix according to claim 5.

27. The kit of claim 24 further comprising an effective amount of target cells selected from RPE, umbilical cells, placental cells, adult stem cells, cells differentiated from adult stem cells, ES cells, cells differentiated from ES cells, bone marrow-derived stem cells, fetal RPE, adult iris pigment epithelial (IPE) cells, Schwann cells, and any combination thereof, derived from autologous or allogenic source.
